(19) United States
(12) Patent Application Publication
(10) Pub. No.: US 2020/0390359 A1
(43) Pub. Date: Dec. 17, 2020

(54) MUSCLE FASCICLE MEASUREMENT

(71) Applicants: Meshal Alhathal, Kearny, NJ (US); Maryam Alkhaldi, Kearny, NJ (US)

(72) Inventors: Meshal Alhathal, Kearny, NJ (US); Maryam Alkhaldi, Kearny, NJ (US)

(21) Appl. No.: 16/440,606

(22) Filed: Jun. 13, 2019

(51) Int. Cl. A61B 5/0488; A61B 5/0492; A61N 1/04; A61B 5/04

(52) U.S. Cl. CPC A61B 5/0488; A61B 5/0492; A61N 1/0452; A61B 5/04012; A61N 1/36014; A61N 1/0476; A61N 1/0456; A61B 2562/14; A61B 2562/043; A61B 5/04001

(57) ABSTRACT

The present disclosure is directed to muscle fascicle measurement systems and methods. A measurement device with electrodes placed in proximity to a target muscle of a subject's body. Stimulating signals may be transmitted by first electrodes placed along a muscle fascicle belonging to a particular muscle and the muscle response is sensed by second electrodes placed in proximity to the particular muscle. Signals sensed by the second electrodes are processed to determine one or more characteristics of the particular muscle fascicle and/or one or more characteristics of a nerve associated with the muscle fascicle.

### TECHNICAL FIELD

Embodiments of the disclosure relate generally to muscle fascicle measurement, and more specifically, relate to determining muscle fascicle characteristics.

### BACKGROUND

A muscle fascicle, or bundle of skeletal muscle fibers, can be examined to determine certain characteristics (e.g., activity, fascicle length, pennation angles, thickness, etc.) to determine whether a patient's muscle is functioning properly or otherwise diagnose a medical issue. Health practitioners use an electromyography (EMG) test to evaluate muscle health and a nerve conduction velocity (NCV) test to evaluate the nerve health associated with the muscle. In some cases, a needle is inserted into the patient's muscle to test the muscle health as part of the invasive EMG test. In other cases, surface EMG is employed, which is a non-invasive procedure that utilizes electrodes to send pulses to muscles and receive feedback.

Conventionally, health practitioners apply gel to patients (e.g., during a non-invasive measurement session), specifically around the treatment area to moisturize the skin and reduce the skin resistivity. Skin resistivity affects the accuracy of measurements during tests, and efficiency when an external pulse is applied.

Currently, health professionals place the electrodes on the targeted area based on their experience about the anatomy and patients' conditions. Moreover, placement of the electrodes can also vary from session to session. When users need to use the electrodes outside of the practitioner's supervision, users have to estimate or guess where to optimally place the electrodes. Furthermore, placing electrodes correctly is especially important when practitioners are targeting specific muscle fibers. Since each patient's condition is different, general guidelines cannot assume pre-defined muscle fibers locations and thus still need to rely on the practitioners' experiences. Some conventional devices include electrodes with pre-defined locations (e.g., by sewing in place or using precut holes to a flexible object or a cloth), which reserves each electrode in place. However, it is difficult to set-up and reconfigure. Furthermore, in such cases, health practitioners are limited to taking measurements according to the pre-defined locations.

### BRIEF DESCRIPTION OF THE DRAWINGS

The present disclosure will be understood more fully from the detailed description given below and from the accompanying drawings of various embodiments of the disclosure.

FIG. 1 depicts a system for determining muscle fascicle characteristics according to embodiments of the disclosed subject matter.

FIG. 2 is an example device for determining muscle fascicle characteristics on a user's arm according to embodiments of the disclosed subject matter.

FIG. 3 depicts examples of various implementations of electrodes used in a device for determining muscle fascicle characteristics, according to embodiments of the disclosed subject matter.

FIG. 4 illustrates an example device for determining muscle fascicle characteristics of a user's arm, according to embodiments of the disclosed subject matter.

FIG. 5 depicts muscle fibers and fascicles measured according to embodiments of the disclosed subject matter.

FIG. 6 illustrates an example microcontroller of a device for determining muscle fascicle characteristics according to embodiments of the disclosed subject matter.

FIG. 7 illustrates a method of determining muscle fascicle characteristics according to embodiments of the disclosed subject matter.

FIG. 8 depicts a timing diagram of transmitted signals according to embodiments of the disclosed subject matter.

FIG. 9 illustrates a method of dispensing gel according to embodiments of the disclosed subject matter.

FIG. 10 is a block diagram of an example gel dispensing system, according to embodiments of the present disclosure.

FIGS. 11A-11E illustrate a locking sub-system and associated stages of operation relating to positioning an electrode and transitioning between a locked and unlocked state, according to embodiments of the present disclosure.

FIGS. 12A-12E illustrates stages of operation of an exemplary handle of an electrode placement system, according to embodiments of the present disclosure.

FIG. 12B depicts lock and unlock handles of an electrode placement system, according to embodiments of the present disclosure.

FIG. 13 is a block diagram of an example computer system that may perform one or more of the operations described herein, in accordance with various implementations.

Embodiments of the disclosed subject matter are described more fully hereinafter with reference to the accompanying drawings. The disclosed subject matter may, however, be embodied in many different forms and should not be construed as limited to the illustrated embodiments. In the drawings, the size and relative sizes of layers and regions may be exaggerated for clarity. Like reference numerals in the drawings denote like elements.

### DETAILED DESCRIPTION

Aspects of the present disclosure are directed to systems and methods for muscle fascicle measurement. According to embodiments of the present disclosure, muscle fascicle measurement can be identified using a measurement device with electrodes placed on one or more muscle of a user's body. The electrodes may include electrical muscle stimulation (EMS) electrodes, transcutaneous electrical nerve stimulation (TENS) electrodes, and Surface Electromyography (sEMG) electrodes. Stimulating signals may be transmitted by the TENS/EMS electrodes placed along a muscle fascicle belonging to a particular muscle to produce a muscle response that is sensed by the sEMG electrodes placed on the particular muscle. Signals sensed by the sEMG electrodes may be further processed to determine characteristics of the muscle fascicle on which the measurement device is placed and a nerve associated with the muscle fascicle. Additionally, embodiments of the disclosure enhance the environment surrounding the muscle fascicles measurements by adding localization and gel dispensing mechanisms. Hereinafter, embodiments of the disclosed subject matter are described in detail with reference to the accompanying drawings.

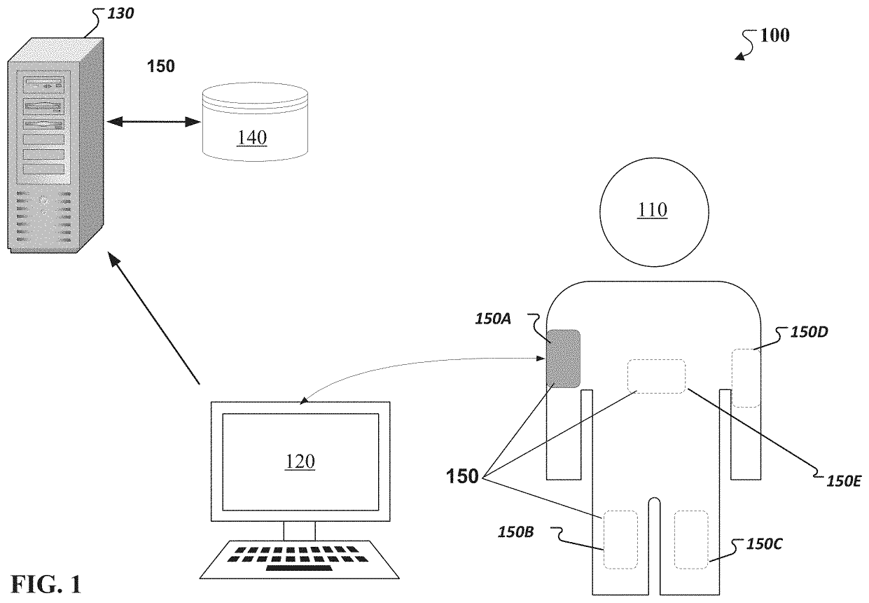

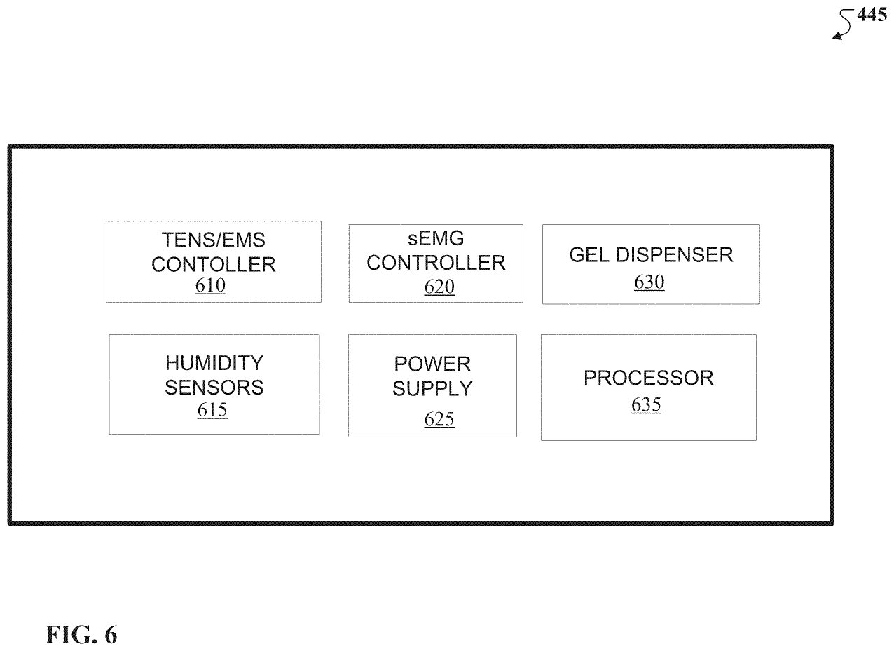

FIG. 1 depicts a system 100 for determining muscle fascicle characteristics according to embodiments of the present disclosure. The system 100 may include a computing device 120, a server 130, a database 140, and a device 150 (hereinafter referred to as “measurement device”) for determining muscle fascicle characteristics of a user or subject 110. The user 110 may be a person or an animal. In an embodiment, the measurement device may be attached to one or more parts of the body of the user 110. For example, the measurement device 150A/B/C/D/E may be attached to the right arm of the user 110, a chest of the user 110, a left arm of the user 110, a right leg of the user 110, a left leg of the user 110, or other areas that need measurements. For simplicity and ease of understanding, the measurement device 150 can include one or measurement device components 150A, 150B, 150C, 150D, and 150E, as shown in the example depicted in FIG. 1. Unless explicitly stated, descriptions of the measurement device component 150A apply to measurement device components 150B/C/D/E placed on one or more areas or parts of the user 110. The measurement device component 150A is described in further detail with reference to FIG. 6.

In general, the computing device 120, network server 130, database 140, and measurement device 150 may be connected to each other through one or more networks. The one or more networks may include and implement commonly-defined network architectures including those defined by standards bodies, such as the Global System for Mobile communication (GSM) Association, the Internet Engineering Task Force (IETF), and the Worldwide Interoperability for Microwave Access (WiMAX) forum. For example, the one or more networks may implement one or more of a GSM architecture, a General Packet Radio Service (GPRS) architecture, a Universal Mobile Telecommunications System (UMTS) architecture, and an evolution of UMTS referred to as Long Term Evolution (LTE). The one or more networks may implement a WiMAX architecture defined by the WiMAX forum or a Wireless Fidelity (WiFi) architecture. The one or more networks may include, for instance, a local area network (LAN), a wide area network (WAN), the Internet, a cloud network that provides Internet services and other network-related functions, e.g., storing data, a virtual LAN (VLAN), an enterprise LAN, a layer 3 virtual private network (VPN), an enterprise IP network, or any combination thereof. The one or more networks may include access points, storage systems, cloud systems, modules, one or more databases including user database 140, and one or more servers including network server 130.

The computing device 120 may be connected to the measurement device 150A and server 130 wirelessly or through a wired connection. For example, in some embodiments, the computing device 120 and the measurement device 150 may be connected through one or more short-range wireless networks such as Bluetooth, Infrared, or Zigbee networks. In some embodiments, wired connections such as optical cables, fiber optic cables, universal serial bus (USB) cables, or conductive wires may be used to transport data between the computing device 120, measurement device 150, and/or the server 130.

In general, the computing device 120 may be any suitable electronic device capable of communicating with other electronic devices through wired or wireless networks. Examples of the computing device 120 include, but are not limited to, a laptop, a desktop, an electronic pad, a mobile phone, a smart phone, a smart television, and a personal digital assistant. The computing device 120 may include input/output interfaces, display devices, storage devices, processors, and other computer components for executing operations performed by the computing device 120 according to the embodiments. For example, the storage device may store code for an algorithm containing instructions, which when executed by the processor, cause the computing device to perform one or more operations.

The network server 130 may include any suitable computing device coupled to the one or more networks, including but not limited to a personal computer, a server computer, a series of server computers, a mini computer, and a mainframe computer, or combinations thereof. The network server 130 may also include a web server, or a series of servers, running a network operating system, examples of which may include but are not limited to Microsoft® Windows® Server, Novell® NetWare®, or Linux®. The network server 130 may be used for and/or provide cloud and/or network computing. Although not shown in the figures, the server may have connections to external systems providing messaging functionality such as e-mail, SMS messaging, text messaging, and other functionalities, such as advertising services, search services, etc.

In some implementations, the network server 130 may send and receive data using any technique for sending and receiving information including, but not limited to, using a scripting language, a remote procedure call, an email, an application programming interface (API), Simple Object Access Protocol (SOAP) methods, Common Object Request Broker Architecture (CORBA), HTTP (Hypertext Transfer Protocol), REST (Representational State Transfer), any interface for software components to communicate with each other, using any other known technique for sending information from a one device to another, or any combination thereof.

System 100 may also include user database 140, which may include a cloud database or a database managed by a database management system (DBMS). A DBMS may be implemented as an engine that controls organization, storage, management, and retrieval of data in a database. DBMSs may provide the ability to query, backup and replicate data, enforce rules, provide security, do computation, perform change and access logging, and automate optimization. A DBMS may typically include a modeling language, data structure, database query language, and transaction mechanism. The modeling language may be used to define the schema of each database in the DBMS, according to the database model, which may include a hierarchical model, network model, relational model, object model, or some other applicable known or convenient organization. Data structures may include fields, records, files, objects, and any other applicable known or convenient structures for storing data. A DBMS may also include metadata about the data that is stored.

In some embodiments, the user database 140 may include profiles of different users in which user information may be stored. For example, the user database 140 may store data indicative of user 110's medical history, data obtained from the measurement device 150A, demographic information, family medical history and additional information, such as, for example, the name, age, and address of the user 110. Data stored in the user database 140 may be treated in one or more ways before it is stored or used, so that personally identifiable information may be removed if desired by the user. For example, user 110 may specify the type of user information that can or cannot be stored in the user database 140. As a result, user information that does not conform to the user 110 approved information need not be stored in the user database 140 or is removed from the user database 140. In an embodiment, the user information may be stored in a manner such that personally identifiable information is approved by the user 110 for storage in his or her respective profile in the user database 140.

In some embodiments, information may be abstracted rather than removed from the user database 140 in response to user selection of information to omit. For example, if a user does not want to specify the user's zip code of 22310, the user's zip code may be abstracted to be “Virginia.”

As described in further detail below, the system 100 shown in FIG. 1 may permit the one or more measurement device components (e.g., 150A, 150B, 150C, 150D, 150E) of measurement device 150 (collectively referred to as the “measurement device 150”) to take measurements to determine muscle fascicle characteristics of the user 110. The muscle fascicle characteristics may be determined by the measurement device 150, and data describing the user 110 and muscle fascicle characteristics of the user 110 may be stored in the user database 140.

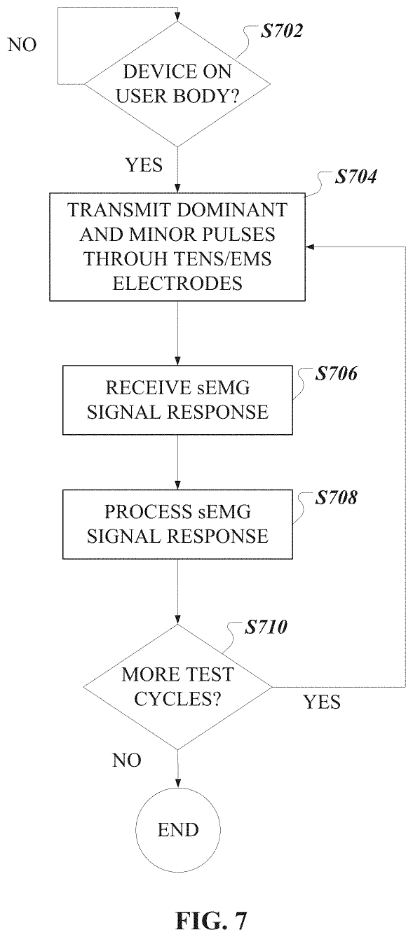

According to embodiments, certain data processing operations described below in connection with FIGS. 7-13 below can be implemented by the measurement device 150 (e.g., the processor 635 of measurement device 150A, as shown in FIG. 6). In some embodiments, one or more of the data processing operations described herein may be executed by the computing device 120 and/or the network server 130. For example, data obtained by the measurement device 150 may be transmitted to the computing device 150A and/or the network server 130 for processing to characterize the muscle fascicles of the user 110. One or more portions of the processed data and, in general, any user information may be stored in the user database 140.

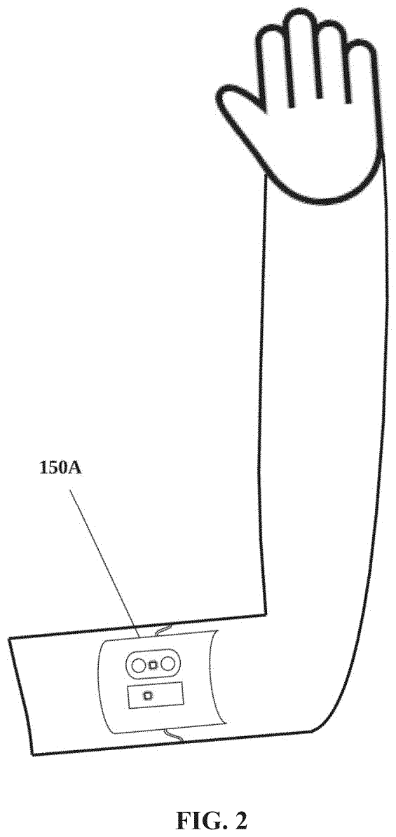

FIG. 2 depicts an image of the measurement device component 150A when wrapped around the user 110's arm. The measurement device 150A may be wrapped around the user 110's arm in several different ways. For example, in some instances, the measurement device 150A may be affixed to a Velcro strap, which may be wrapped around or attached to an arm of the user 110 as shown in FIG. 2, or, more generally, any part of the user 110's body.

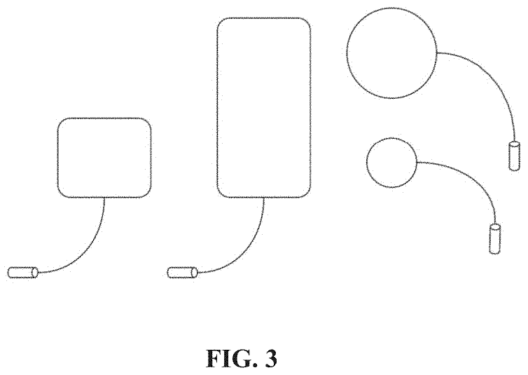

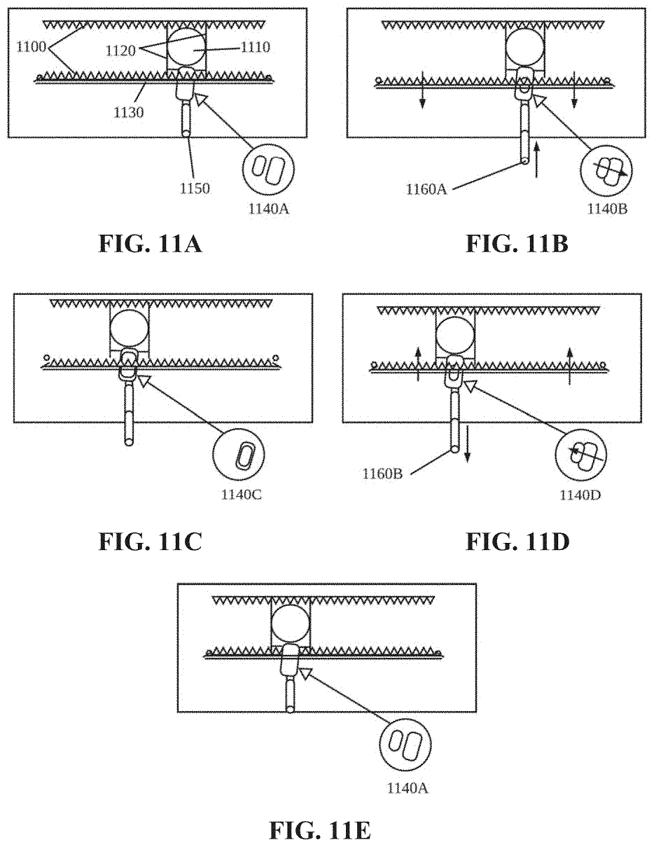

The measurement device 150A and components within the measurement device 150A may be implemented in various different shapes and sizes. As shown in FIG. 3, electrodes in the measurement device 150A may have any suitable shape, including a rectangular, square, or circular shape. The shape and size of the electrodes may vary based on the part of a user 110's body on which the measurement device 150A is to be attached. For example, the electrodes may have an elongated, rectangular shape and be utilized to determine calf muscle characteristics. Another electrode may have a square shape and be utilized to determine characteristics of a chest muscle such as the pectoralis major. Additionally, any electrode may include underneath a mechanism to block movements in any direction, as shown in FIGS. 11A/D. In FIGS. 11A/D, any electrode may be placed in between zig-zag tracks 1100 and 1140 in order hold its location. In FIGS. 11B/C, any electrode can be unlocked by extending the distance between the tracks 1100 and 1140, enabling electrodes to move vertically using a handle 1170.

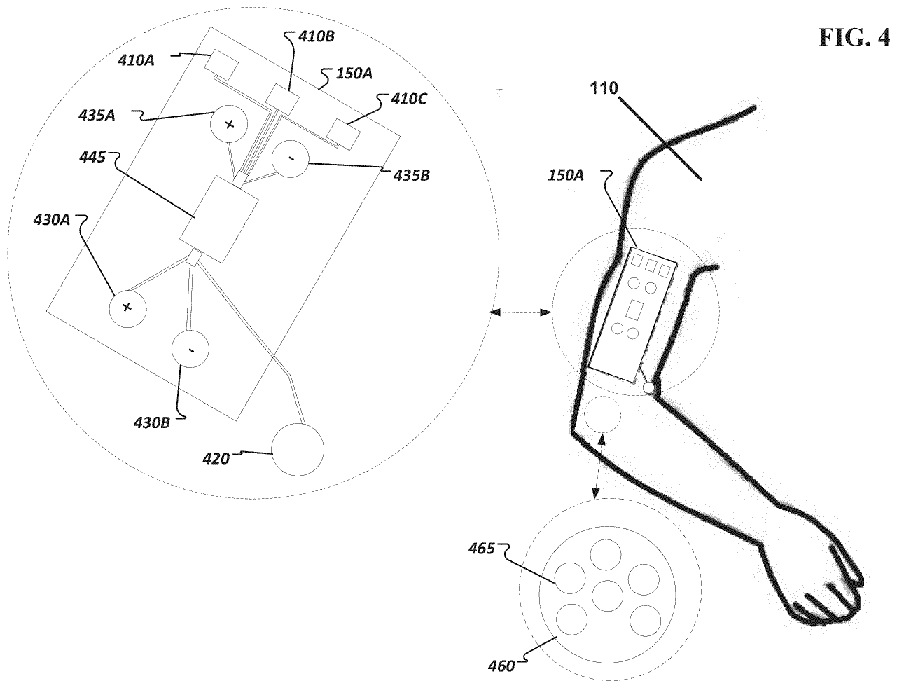

In an embodiment, a power cable may be connected to the measurement device 150A to provide supply voltage and current to the electronic components within the measurement device 150A. Details of the electronic components are described with respect to FIGS. 4 and 6. As shown in FIG. 4, the measurement device 150A may include stimulating electrodes 410A, 410B, and 410C, sensing electrodes 435A, 435B, 430A, and 430B, a reference electrode 420, and a microcontroller 445.

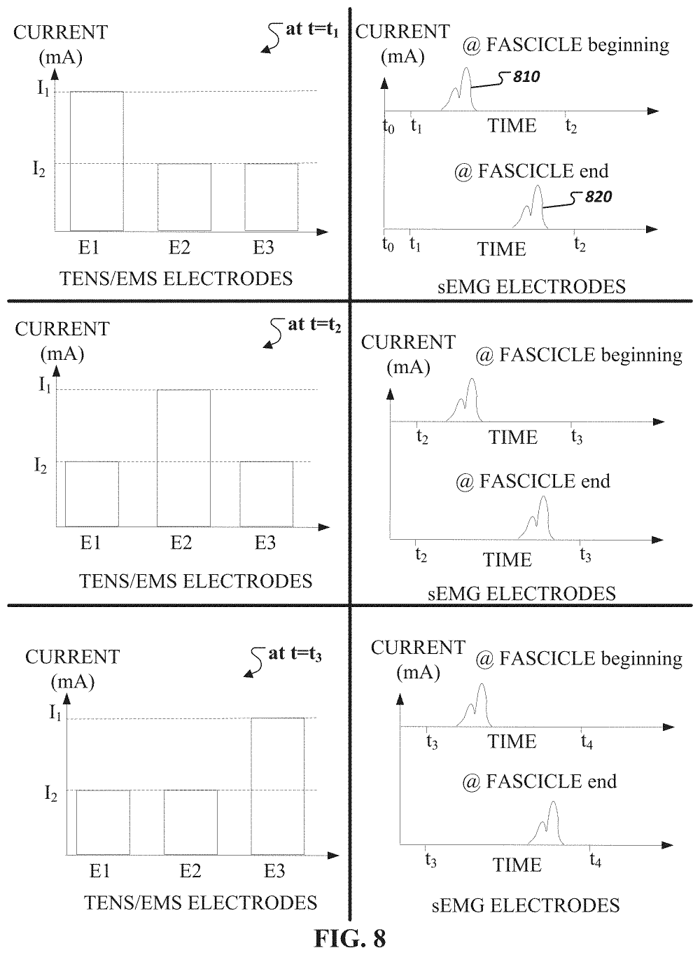

In some embodiments, the stimulating electrodes 410A, 410B, and 410C may be EMS and/or TENS electrodes that are placed on the skin of the user 110. In particular, each of the stimulating electrodes 410A, 410B, and 410C may be placed on different muscle fascicles of the same muscle group. With reference to FIG. 6, the EMS/TENS electrodes may be connected to and controlled by the EMS/TENS controller 610 in a microcontroller (e.g., microcontroller 445 of FIG. 4). The EMS/TENS controller 610 may control the strength and timing of signals transmitted by the stimulating electrodes 410A, 410B, and 410C. For example, the EMS/TENS controller 610 of FIG. 6 may provide a dominant signal in an alternating manner to each of the stimulating electrodes 410A, 410B, and 410C of FIG. 4. Non-dominant signals may be transmitted to any of the stimulating electrodes 410A, 410B, and 410C that is not receiving a dominant signal. Further details of the signals provided to the stimulating electrodes 410A, 410B, and 410C are provided with respect to FIG. 8 below. Although only three stimulating electrodes are shown in FIG. 4, in general, any suitable number of stimulating electrodes greater than one may be used.

In some embodiments, the sensing electrodes 435A, 435B, 430A, and 430B may be sEMG electrodes that are placed on the skin of the user 110, e.g., on different parts of the muscle group on which the stimulating electrodes 410A, 410B, and 410C were disposed. The sEMG electrodes 435A, 435B, 430A, and 430B may be connected to and controlled by the sEMG controller 620 of FIG. 6 (e.g., in microcontroller 445 of FIG. 4). The sEMG controller 620 may receive data indicative of sensed signals from the one or more sensing electrodes (e.g., sensing electrodes 435A, 435B, 430A, and 430B of FIG. 4).

In some embodiments, the sensing electrodes 435A, 435B, 430A, and 430B may be divided into pairs, such as the sensing electrode pair 435A and 435B, and sensing electrode pair 430A and 430B. A voltage potential difference may exist between electrodes in each pair of sensing electrodes when a signal is detected by the sensing electrode pair. In some cases, each pair of sensing electrodes 435A, 435B, 430A, and 430B may be arranged to be in a linear direction in parallel to the direction that the muscle fascicles in a muscle extend. In general, each electrode in a pair of sensing electrodes 435A, 435B, 430A, and 430B may be located on any part of a muscle group and is spaced apart from the other electrode in the pair by a determined distance, e.g., approximately 40 mm.

In general, when signals are transmitted by stimulating electrodes 410A, 410B, and 410C, the signal(s) may be transmitted through one or more nerves along the muscle fascicles on which the stimulating electrodes 410A, 410B, and 410C are placed. Transmission of the one or more signals along one or more nerves in a muscle may cause the muscle to contract, thereby generating electrical activity within the muscle, which may be measured by the sensing electrodes 435A, 435B, 430A, and 430B. Since sensing electrodes 435A and 435B may be located closer to the stimulating electrodes 410A, 410B, and 410C than sensing electrodes 430A and 430B, sensing electrodes 435A and 435B may detect an electrical signal earlier than sensing electrodes 430A and 430B. In an embodiment, the time difference between when the electrical signal is detected at sensing electrodes 435A and 435B and sensing electrodes 430A and 430B may be an indication of how long a signal takes to travel through a nerve associated with the stimulated muscle fascicles.

In some embodiments, the measurement device component 150A may also have a reference electrode 420. The reference electrode 420 may be placed on the user 110 in an area away from the muscle fascicle or muscle group for which characteristics are being determined. For example, if the measurement device 150A is being used to measure characteristics of a calf muscle on the right leg, the reference electrode 420 may be placed on a muscle other than the calf muscle on the right leg. The reference electrode 420 may sense background noise, e.g., noise arising from a heartbeat or movement of the user 110, when measurements are being obtained by the measurement device component 150A. The background noise may be used to remove noise and interferences in the sEMG signals detected by any of the sEMG electrodes 435A, 435B, 430A, and 430B. In the example shown in FIG. 4, the measurement device 150A is disposed on the skin of the user 110 in an area corresponding to the bicep muscle area, and the reference electrode 420 may be disposed on the skin of the user 110 in an area corresponding to a brachialis muscle.

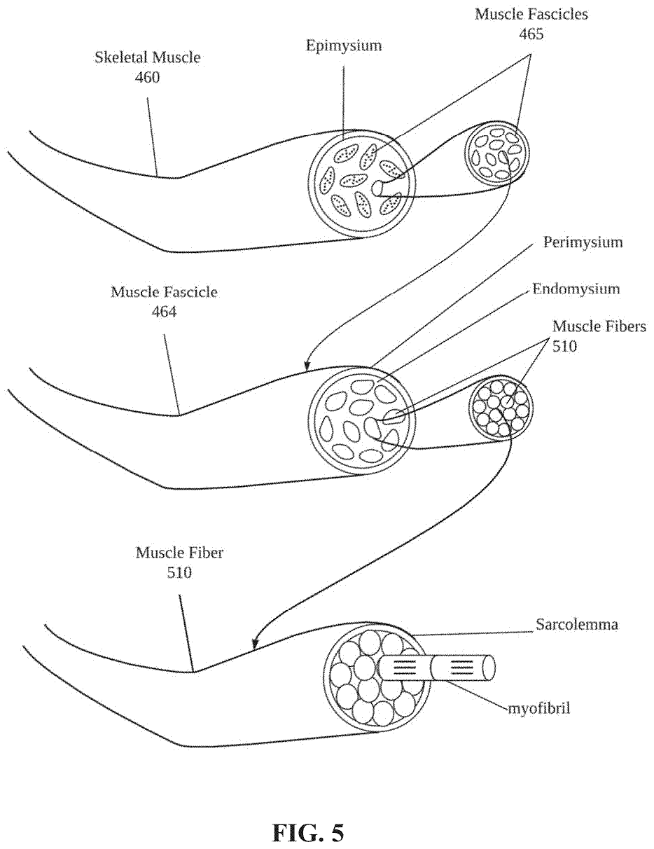

FIG. 5 depicts a diagram of example muscle fibers and fascicles. An example muscle group or skeletal muscle 460 is shown. Each muscle group 460 may include blood vessels and a plurality of muscle fascicles 465 surrounded by fibrous muscle tissue, known as epimysium. Each muscle fascicle 465 may include a plurality of muscle fibers 510, perimysium, and endomysium. The endomysium is a layer of areolar connective tissue that covers and protects each individual muscle fiber 510 and contains capillaries and nerves. The endomysium overlies the cell membrane of muscle fiber 510. The perimysium is the sheath of connective tissue surrounding a bundle of muscle fibers. In an example, each of the stimulating electrodes 410A, 410B, and 410C may be disposed on skin directly above an individual muscle fascicle. The stimulating electrodes 410A, 410B, 410C may therefore be separated by the distance of the respective muscle fascicles on which the stimulating electrodes 410A, 410B, 410C are disposed. Each muscle fiber 510 may have a cell membrane, known as sarcolemma, consisting of a plasma membrane and an outer coat consisting of a thin layer of polysaccharide material. Each muscle fiber 510 may also include myofibril and a nucleus.

Referring back to FIG. 4, the measurement device component 150A may also include a microcontroller 445 that is connected to and communicates with each of the electrodes 410A, 410B, 410C, 420, 430A, 430B, 435A, and 435B. The measurement device 150A may include electronic paths or conductive wires that allow the microcontroller 445 to transmit and receive electronic signals to and from the electrodes 410A, 410B, 410C, 420, 430A, 430B, 435A, 435B.

In addition, in some embodiments, the measurement device 150A may include gel pathways that allow gel to be dispensed at an area corresponding to the electrodes 410A, 410B, 410C, 420, 430A, 430B, 435A, and 435B. In FIG. 4, the gel pathways and conductive wires are represented by the pair of lines extending from the microcontroller 445 to each of the electrodes 410A, 410B, 410C, 420, 430A, 430B, 435A, and 435B.

Referring to FIG. 6, the microcontroller 445 may include a TENS/EMS controller 610, a sEMG controller 620, a gel dispenser 630, one or more humidity sensors 615, a power supply 625, and a processor 635. As described above, the TENS/EMS controller 610 may transmit current to the TENS/EMS electrodes 410A-C for which the TENS/EMS electrodes 410A-C transmit current into the user 110. The TENS/EMS controller 610 may control the current provided to the TENS/EMS electrodes 410A-C such that the TENS/EMS electrodes 410A-C transmit a dominant signal in an alternating manner.

An example of the alternating dominant signal is shown in FIG. 8. At a particular time (e.g., t₁), the TENS/EMS controller 610 may transmit to the TENS/EMS electrode 410A (E1) a dominant signal having a current I₁. At the same time, the TENS/EMS controller 610 may transmit to each of TENS/EMS electrodes 410B (E2) and 410C (E3) a non-dominant (minor) signal having a current I₂. The power ratio of the dominant signal to the minor signal may be used for data processing and may range, for example, from one to 250. The dominant signal and the minor signal may be pulse signals having a particular frequency, for example, between 50 Hz and 200 Hz.

After a period of time, a second cycle may be initiated and the dominant and minor signals may be transmitted again. However, the electrodes transmitting the dominant and minor signals vary. For example, at the second time, e.g., t₂, the TENS/EMS controller 610 may transmit to the TENS/EMS electrode 410B (E2) a dominant signal having a current I₁, and transmit to TENS/EMS electrodes 410A (E1) and 410C (E2) a minor signal having a current I₂.

After another period of time, a third cycle may be initiated and the dominant and minor signals may be transmitted again. However, the electrodes transmitting the dominant and minor signals may vary. For example, at the third time, e.g., t₃, the TENS/EMS controller 610 may transmit to the TENS/EMS electrode 410C (E3) a dominant signal having a current I₁, and transmit to TENS/EMS electrodes 410A (E1) and 410B (E2) a minor signal having a current I₂.

This alternating process may continue until each TENS/EMS electrode 410C has transmitted a dominant signal at least once or until an operator or processor 635 of the measurement device 150A terminates measurements operations.

As noted above, the sEMG electrodes 430A, 435A, 430B, and 435B may sense a signal when a muscle contracts in response to stimulation by the TENS/EMS electrodes 410A-C. For example, a pair of sEMG electrodes 435A and 435B is placed at a beginning portion of a muscle fascicle in close proximity to TENS/EMS electrodes 410A-C. In this example, a dominant stimulating signal may be transmitted by TENS/EMS electrode 410A at time t₁, and an electrical signal 810 may be detected by sEMG electrodes 435A and 435B shortly after time t₁ at the beginning of the muscle fascicle, as shown in FIG. 8. In this example, no signal may be detected by sEMG electrodes 430A and 430B shortly after time t₁ at the end of the muscle fascicle.

After another short period of time though, the stimulation by the TENS/EMS electrodes 410A-C may traverse through the muscle and nerves associated with the muscle, and the other pair of electrodes 430A and 430B located at a portion (e.g., an end) of the muscle fascicle may detect a signal 820. In FIG. 8, this detected signal 820 may be shown by the increase in voltage detected at the fascicle end by sEMG electrodes shortly after time t₁ but before time t₂.

According to embodiments, the signals detected by the sEMG electrodes 430A, 435A, 430B, and 435B may have a number of peaks and dips. The peaks and dips may be attributed to a cumulative response of a muscle to the stimulation by the TENS/EMS electrodes 410A-C. For example, nerves associated with muscle fascicles located at different distances from the sEMG electrodes 430A, 435A, 430B, and 435B are stimulated by different currents, i.e., dominant and minor signals. These different currents may generate different responses from the muscle. For example, in some cases, an electrical signal generated in response to the TENS/EMS stimulation has an amplitude that is smaller than the largest stimulating signal. In some cases, an electrical signal that is even larger than the stimulating currents may be generated by the muscle in response to the TENS/EMS stimulation. Referring to FIG. 8, the larger peak voltage detected after a smaller peak voltage at a beginning portion of the fascicle may be attributed to the muscle response to the dominant signal. In general, the signals detected by the sEMG electrodes 430A, 435A, 430B, and 435B may be low power and low frequency signals.

The above-described TENS/EMS stimulation and sEMG detection operations may be performed each time a TENS/EMS stimulating signal is detected by a sEMG electrode. As shown in FIG. 8, in addition, a sEMG signal may be transmitted after time t₂ and t₃.

As illustrated in FIG. 6, the sEMG controller 620 may receive data indicative of the signals detected at the sEMG electrodes (e.g., electrodes 430A, 435A, 430B, and 435B of FIG. 4). In some embodiments, the sEMG controller 620 may perform one or more signal processing operations after receiving the data. For example, the sEMG controller 620 may perform filtering, amplifying, and/or analog-to-digital conversion operations on the received data. In some embodiments, the sEMG controller 620 may also perform a subtraction operation in which the TENS/EMS stimulating signals are subtracted from the signals received at the pair of sEMG electrodes 430A and 430B or 435A and 435B. In an example, the power in a sEMG signal may be substantively less than a TENS/EMS stimulating signal, e.g., 10-100 times less power than a TENS/EMS stimulating signal. Accordingly, the subtraction operation may be performed after the signal received by the sEMG electrodes 430A, 435A, 430B, and 435B is amplified. In some embodiments, the sEMG controller 620 may forward data received from the sEMG electrodes 430A, 435A, 430B, and 435B to processor 635, which may perform one or more of the above-noted sEMG signal processing steps.

In an embodiment, processor 635, shown in FIG. 6, may be coupled to one or more of the components of the microcontroller 445, and may control the operations of the microcontroller 445. The processor 635 may receive data from and transmit data to each component of the microcontroller 445, and may control the transfer of data from one component of the microcontroller 445 to another. The processor 635 may include various logic circuitry and execute programs and algorithms to implement the various embodiments described herein. For example, the processor 635 may be connected to a clock or timer, and may send instructions to the TENS/EMS controller 610 at certain times that are synchronized with the clock/timer to control the transmission of signals by the TENS/EMS electrodes 410A-C. Additional descriptions of the processor 635 and hardware and software configurations of the processor 635 are provided below.

A power supply 625 may be included in the microcontroller 445 and may be configured to provide power to each component of the microcontroller 445. In general, various suitable types of power supplies may be used. For example, in some embodiments, the power supply 625 may be connected to a power supply cable that provides external power to the microcontroller 445. In some embodiments, the power supply 625 may be a battery such as a cell battery, an alkaline battery, a lithium ion battery, a nickel metal battery, a mercury battery, a silver oxide battery, or a zinc air battery.

In some embodiments, the microcontroller 445 may also include or be connected to one or more humidity sensors 615. A humidity sensor 615 may be configured to detect a humidity level where the measurement device component 150A is disposed. For example, the humidity sensor 615 may sense the amount of humidity on the skin surface of a user 110 on which the measurement device 150A is placed on. In general, various types of humidity sensors may be used. For example, the humidity sensor 615 may include, but is not limited to, a capacitive humidity sensor, a resistive humidity sensor, or a thermal conductivity humidity sensor.

In some embodiments, the humidity sensor 615 may be configured to obtain humidity measurements periodically, and, in some embodiments, the humidity sensor 615 may obtain humidity measurements in response to instructions received from the processor 635.

In some embodiments, a plurality of the humidity sensors 615 may be dispersed throughout the measurement device 150A such that each humidity sensor is located in close proximity to or within a particular distance of an electrode. In such cases, the humidity sensors 615 may provide the resolution to differentiate between the humidity levels at different electrodes. In some embodiments, one or more humidity sensors may be located in a central location of the measurement device 150A so that the average or general humidity level between the measurement device 150A and the skin of the user 110 may be determined. Data indicative of the humidity levels detected by the humidity sensors 615 may be transmitted to the processor 635.

The microcontroller 445 may also include or be connected to a gel dispenser 630. The gel dispenser 630 is configured to dispense gel in the space between the electrodes and the skin of the user 110. The gel dispenser 630 may be connected to a plurality of gel tubes that transfer gel from the gel dispenser 630 to the skin surface of the user 110. The processor 635 may receive data indicative of humidity levels detected by the one or more humidity sensors 615, and, in response to receiving the humidity level data, the processor 615 may instruct the gel dispenser 630 to dispense gel at certain locations.

In some embodiments, the microcontroller 445 may connect to the computing device 120 to allow practitioners to configure the gel dispenser 630. Practitioners may customize the gel dispensing periods by interacting with a graphical user interface of the computing device 120 while a user is exercising. Practitioners may also customize the gel dispensing periods by recording a user and selecting the dispensing periods based on a review of the recording. For example, after the video is recorded, a practitioner may play back the video and choose a specific time, say at the 1:00 minute mark, to click on a button or move a slider. These input methods are then used to set the desired time(s) according to the video length and gel amounts to be sent by the server 130. The times and amounts may be saved on the device's memory or a database for reference and use in one or more subsequent sessions. In an embodiment, when a user begins a new session, previously stored commands, selections, options relating to the dispensing of gel are retrieved, including the corresponding dispensing times and gel amounts.

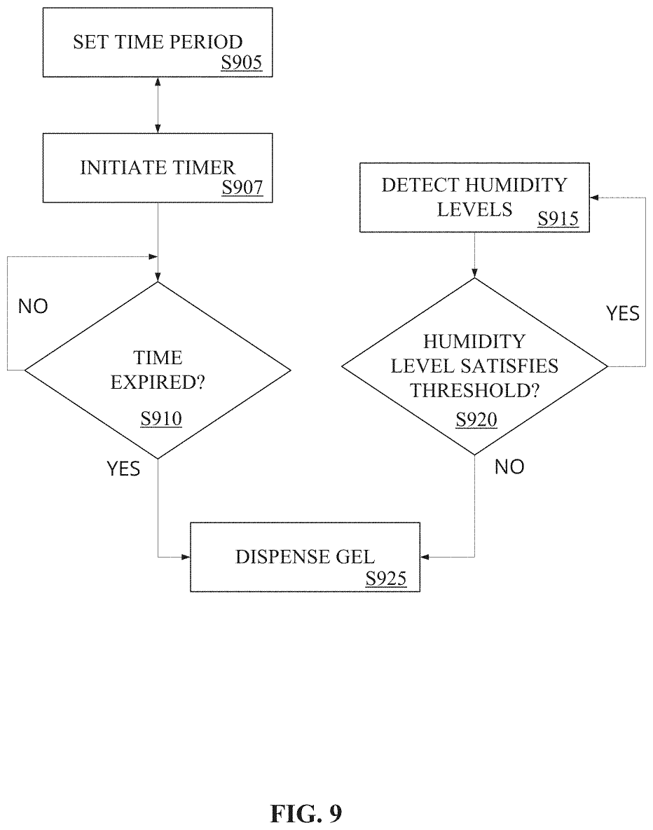

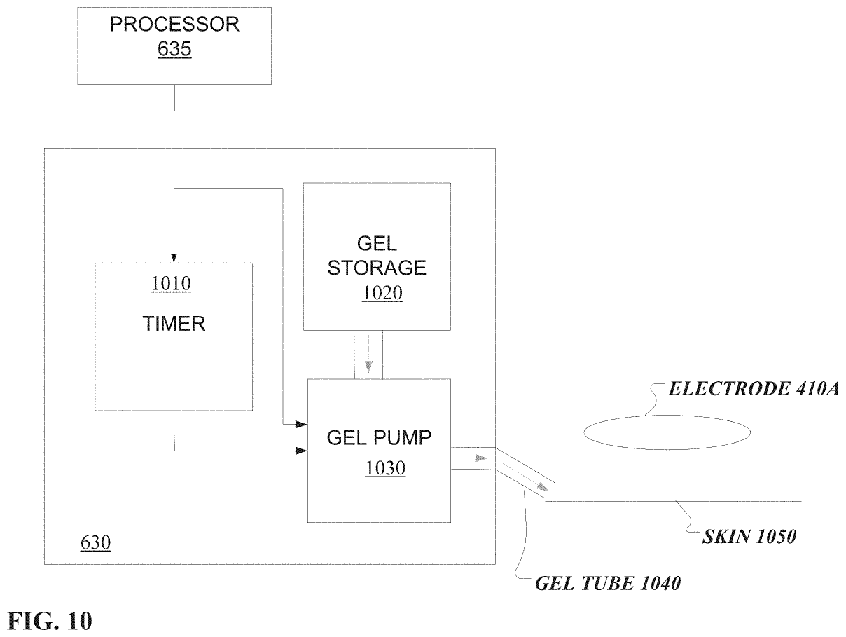

In general, various suitable types of conductive gel may be used. For example, gel containing propylene glycol, glycerine, perfume, dyes, phenoxyethanol, carbapol R 940 polymer, water, and sodium chloride may be used. The gel dispenser 630 and gel dispensing process are described further with reference to FIGS. 9 and 10.

Placement of the electrodes in a correct, proper or optimized position improves the accuracy of measurements. In an embodiment, a practitioner can set up the system described herein during an initial phase, and the system is configured to store the setting established during the initial phase. In an embodiment, the settings may be directed to lock the placements of the electrodes in order to identify and target one or more particular muscle fibers, as shown in FIGS. 11A-D. In an embodiment, the settings may be directed to the gel dispensing periods, as shown in FIGS. 6/7/10. In an embodiment, the system may be configured to collect one or more data points associated with various conditions to enable the system to record or store placement and gel dispensing information for the electrodes without further inputs from a practitioner.

In an embodiment, electrodes such as 430A/B and 435A/B may include an inertia measurement unit (IMU) to calculate the x-y-z locations. An IMU may be included on each electrode so that the processor 635 may send the locations of the electrodes for the computing device 120, processor 635, and/or database 140. Furthermore, the IMU readings may go into an initial stage to get the expected values for the locations of the electrodes. In the initial stage, a processor 635, a computing device 120, or a database 140 may take the initial x-y-z values of each electrodes and store the values for subsequent use (e.g., the initial values may be used as a comparison for subsequent electrodes placements). In an embodiment, in response to a user setting up a subsequent session, the processor and/or computing device may perform calculations to inform a user when the electrodes are significantly misplaced as compared to the placement associated with the initial stage.

FIG. 7 illustrates an example method executable by a measurement device (e.g., measurement device 150 of FIG. 1) to determine various characteristics of a muscle fascicle. As described below, the characteristics include a quality of a muscle fascicle and/or a responsiveness of a nerve associated with the muscle fascicle.

To begin measuring muscle fascicle characteristics, the measurement device 150A determines whether the measurement device 150A has been placed on a part of the user 110's body (S702). For example, the measurement device 150A may include one or more sensors, such as capacitive sensors, light sensors, resistance sensors, to detect when the measurement device 150 is in contact with the skin of a user 110. Upon sensing contact with the user 110's skin, the one or more sensors may send a signal to the processor 635 of the measurement device 150A to indicate that the measurement device 150A is placed on the user 110's body.

The measurement device 150A may be placed on any muscle of the user 110's body. A person such as a medical practitioner, e.g., nurse, doctor, physical therapist, trainer, athletic coach, etc. may place the measurement device 150A on the user 110 through various methods. For example, the measurement device 150A may be wrapped around a user 110's body part using a belt, Velcro, or placed on the user 110's body using an adhesive.

In some embodiments, each of the TENS/EMS electrodes 410A-C may be positioned on skin directly above different muscle fascicles. For example, TENS/EMS electrode 410A may be placed on or above a first muscle fascicle towards a beginning portion a muscle fascicle. TENS/EMS electrode 410B may be placed on skin directly above a portion of a second muscle fascicle, for example, towards a beginning portion of the second muscle fascicle. TENS/EMS electrode 410A may be separated from TENS/EMS electrode 410B by a distance approximately equal to a distance between the centers of the first and second muscle fascicles that both belong to the same muscle. TENS/EMS electrode 410C may be placed on skin directly above a portion of a third muscle fascicle, for example, towards a beginning portion of the third muscle fascicle.

The sEMG electrodes 430A may be placed on skin above the same muscle group on which the TENS/EMS electrodes 410A-C are disposed. A pair of sEMG electrodes 435A and 435B may be placed closer to the TENS/EMS electrodes 410A-C, e.g., along the beginning portions of the first, second, or third muscle fascicles. A second pair of sEMG electrodes 430A and 430B may be placed further away from the TENS/EMS electrodes 410A-C, e.g., along end portions of the first, second, or third muscle fascicles.

After determining that the measurement device 150A is placed on the user 110's body, the measurement device 150A may activate all the components of the measurement device 150A. After activation, electrical pulses may be transmitted through the TENS/EMS electrodes 410A-C (S704). As described above and shown in FIG. 8, a dominant stimulating signal and minor stimulating signals may be transmitted in an alternating manner. For example, the TENS/EMS controller 610 in microcontroller 445 may transmit a dominant stimulating signal to TENS/EMS electrodes 410A using current I₁ at time t₁, a dominant stimulating signal to TENS/EMS electrodes 410B using current I₁ at time t₂, and a dominant stimulating signal to TENS/EMS electrodes 410C using current I₁ at time t₃. When a TENS/EMS electrode is not transmitting a dominant stimulating signal, it transmits a minor stimulating signal having an amplitude of current I₂.

The stimulating signals transmitted by the TENS/EMS electrodes 410A-C may stimulate one or more nerves along the muscle fascicles above which they are located. The stimulation of the one or more nerves causes the muscle to contract thereby generating electrical activity within the muscle. In S706, this electrical activity may be received or sensed by sEMG electrodes 435A and 435B and sEMG electrodes 430A and 430B at different times, as described above.

At operation S708, the signals detected by the sEMG electrodes 430A, 430B, 435A, and 435B may be provided to the sEMG controller 620 and/or processor 635 for further processing. For example, the processing of the sEMG signal response by the EMG controller 620 and/or processor 635 may include amplifying and filtering the detected signals, and determining the time difference between when a signal was detected by sEMG electrodes 435A and 435B and when a signal was detected by sEMG electrodes 430A and 430B. The time difference may indicate a responsiveness of a nerve associated with the muscle fascicles on which the TENS/EMS electrodes 410A are disposed. A small-time difference may indicate a highly responsive nerve, whereas a large time difference may indicate a nerve that is poorly responsive. The detected time difference may be verified by dividing the distance between the location of electrode pairs 430A/430B and the location of electrode pair 435A/435B, by the expected conduction velocities, e.g., approximately 50-60 meter per second (m/s).

In some embodiments, data in a reference signal received from the reference electrode 420 may also be utilized for improving signal accuracy. For example, background noise, e.g., noise arising from a heartbeat or movement of the user 110, sensed by the reference electrode 420 may be aggregated with the signals detected by the sEMG electrodes 435A, 435B, 430A, and 430B to remove noise and interferences in the signals detected by the sEMG electrodes 435A, 435B, 430A, and 430B.

After processing the signals detected by the sEMG electrodes 435A, 435B, 430A, and 430B, the processor 635 may determine whether another cycle of measurements should be performed to obtain additional samples of signals detected by the sEMG electrodes 435A, 435B, 430A, and 430B, in operation S710. The first cycle, for example, may have been performed with TENS/EMS electrodes 410A-C transmitting dominant and minor pulses at time t₁. If the processor 635 determines that another cycle of measurements should be performed, the TENS/EMS electrodes 410A-C may transmit dominant and minor pulses at subsequent times, e.g., time t₂.

In general, any number of cycles may be performed. In some cases, the person or operator who placed the measurement device 150A on user 110 may terminate the cycles by removing the measurement device 150A. In some cases, cycles may be performed until each TENS/EMS electrode has transmitted a dominant stimulating signal. In some cases, cycles may be performed until a certain number, e.g., 10, of signals detected by the sEMG electrodes 435A, 435B, 430A, and 430B have been processed by processor 635.

In an embodiment, when multiple samples of sEMG detected signals are processed, the processor 635 may average the results as part of the processing operation in S708. For example, if five cycles are executed, the processor 635 may average the determined response time for a muscle fascicle using the five response times determined during each cycle. In this example, in response to a sixth cycle being executed, the average response time may be updated to incorporate the sixth cycle response time in the determined average response time.

In some embodiments, after executing multiple cycles, the processor 635 may further process the signals detected by the sEMG electrodes 435A, 435B, 430A, and 430B to determine characteristics of the muscle fascicles on which the TENS/EMS electrodes 410A-C are disposed. For example, signals detected in response to transmission of dominant and minor stimulating signals may be compared with expected responses to similar dominant and minor stimulating signals transmitted across muscle fascicles having similar structure and length. For example, the signal amplitude or phase and response time detected in response to a dominant stimulating signal across a particular type of fascicle in a particular muscle group (e.g., calf muscle) may be compared to the expected signal amplitude or phase and response time detected in response to a dominant stimulating signal across the same type of fascicle in the same muscle group (e.g., the calf muscle).

In an embodiment, if the comparison indicates that the detected responses to dominant and/or minor stimulating signals have a similarity level that satisfies (i.e., is greater than or equal to) a threshold level, the muscle fascicle may be determined to be healthy and correspond to expected muscle fascicle health. If the comparison indicates that the detected responses to dominant and/or minor stimulating signals have a similarity level that does not satisfy a threshold level, the muscle fascicle may be determined to be unhealthy. In addition, differences between the detected responses to dominant and minor stimulating signals and expected responses may be identified to determine how compromised the health of the muscle fascicle is.

In some cases, a signal may not be detected within a particular time period by sEMG electrodes 430A and 430B in response to a dominant or minor stimulating signal. In some cases, a weak signal may be detected by sEMG electrodes 430A and 430B. A failure to detect a signal or the detection of a weak signal by the by sEMG electrodes 435A, 435B, 430A, and 430B may indicate that the muscle fascicle has some physiological damage or may not be consistent with the anticipated muscle fascicle structure. Accordingly, in the various foregoing manners, indicators of the muscle fascicle health may be determined.

In an embodiment, when no more cycles are to be performed, the measurement device 150A may end the process for obtaining muscle fascicle measurements. Advantageously, non-invasive measurements may be obtained in a simple and cost-effective manner. Furthermore, a user 110 may obtain information about muscle health with an increased muscular resolution (e.g., health at an individual muscle-fascicle level may be determined). For instance, using test results, a medical practitioner may determine the likelihood of muscular damage in a muscle fascicle or the functionality of a muscle fascicle based on the muscle fascicle's responsiveness.

In some embodiments, prior to executing the method illustrated in FIG. 7, the measurement device 150 may be calibrated by executing one or more operations in a calibration mode. For example, the measurement device 150 may transmit test signals through the TENS/EMS electrodes 410A-C, and detect the response to these signals at the sEMG electrodes 435A, 435B, 430A, 430B. In an embodiment, phase and amplitude information of the response to the test signals may be displayed at a display of the measurement device 150A or computing device 120 to allow an operator to adjust positions of the electrodes of the measurement device 150A to positions at which the best signal and least interference is detected by the sEMG electrodes 435A, 435B, 430A, and 430B.

To facilitate the accuracy of measurements obtained by the measurement device 150, a method for automatically dispensing gel between electrodes and the skin of the user 110 may also be executed by the measurement device, as shown in FIGS. 9 and 10. The embodiments associated with method for controlled gel dispensing enables a user or practitioner to employ a gel system capable of determining one or more instances to dispense or apply gel. In an embodiment, the gel dispenser is configured to predict one or more optimizes or desired times to apply a gel and an optimal amount of the gel to dispense. The system may operate based on various settings and may work individually or combined for a more accurate and versatile system. In general, gel dispensing may be performed in one or more of the multiple operation modes, as described in detail below.

In an example, the measurement device 150 can be configured to operate in one of three operation modes, wherein the first operation mode may be based on time, the second operation mode is based on humidity sensors, and the third operation mode is based on syncing gel pumps with exercise patterns.

FIG. 9 illustrates an example method for managing and controlling the dispensing of gel. In a first operation mode, a time period may be set using various suitable methods or user-input on the software or on a device, in operation S905. For example, a user can instruct a system to apply an amount of gel (e.g., 5 milliliters (mL)) at an identified frequency or time period (e.g., every 5 minutes). In an embodiment, the instruction may be received via a computing device 120 or a screen or a knob on a device, such as FIG. 10. To add flexibility, a push button feature may be included to enable a patient or practitioner during the session to pump gel at any time in response to an interaction with the push button or other type of input selection mechanism (e.g., holding a dispense button down to execute the dispensing action). The push button may be on a device, such as on FIG. 10, to control the time and amount of gel or on a connected device, such as a phone, where the button is a binary entry to instruct a system whether to release or hold gel. When the processor 635 receives a push event, it may instruct the gel dispenser 630 to supply gel as long as a user is holding the push button. The embodiment may also benefit from internal or external memory to save data points for future usage. For example, a system may mark the starting and ending time of each session performed by a practitioner and store the durations between each push event. Then, when a user needs to perform those activities, the system retrieves the push events and apply them according to the times and durations obtained before. Also, these push button events may also be a starting point for subsequent training sessions.

In some cases, the time period may be selected or set by receiving an input from an operator of the measurement device 150A to specify a time period between each pump, e.g., 60 seconds, 90 seconds. In some cases, the processor 635 may automatically set an expiration time period according to predetermined rules. For example, the processor 635 may set the time period differently according to the profile of the user 110, e.g., based on a skin type or age of the user 110. Information on the profile of the user 110 may be obtained from the user database 140, as described with reference to FIG. 1. The processor 635 may also synchronize all components of the microcontroller 445 with a clock.

After the time period is set, the processor 635 may transmit instructions to a timer to initiate a time counter, at operation S907. In an embodiment, the timer initiates a new time period upon receiving the instructions. The processor 635 may then determine whether the set time period has expired, at operation S910. In an embodiment, the processor 635 may make this determination by continuously checking the time of the timer. When the time of the timer matches the time-period set in operation S905, the processor 635 may determine that the set time period has expired. The processor 635 may also have a build-in timer, for which the processor needs to check the timing within its logic.

In response to determining that the set time period has expired, the processor 635 may send instructions to the gel dispenser 630 to dispense gel, at operation S925. The gel dispenser 630 may dispense gel on the skin of the user 110, as described in further detail with reference to FIG. 10.

In the second operation mode, the gel dispenser 630 may be configured to dispense gel based on data received from humidity sensors 615. A humidity sensor may be used to detect a humidity level in a surrounding area to predict when the skin needs gel. The humidity sensor unit is relative humidity (RH), typically ranging between 5% to 95%. As described above, one or more humidity sensors 615 may be used to detect the humidity level between the electrodes of the measurement device 150A and the skin of the user 110, at operation S915. In some embodiments, the humidity sensors 615 may be placed in close proximity to the electrodes of the measurement device, for example, within a threshold distance, of the electrodes. In some embodiments, the humidity sensors 615 may be located towards the center of the measurement device 250A. In some embodiments, the humidity sensors 615 may be randomly positioned at different areas between the measurement device 150A and the skin of the user 110. In some embodiments, a single humidity sensor may be used where the readings obtained may be assumed to cover the entire region. Thus, the dispensing time and amount of gel may be one entry for the entire region, e.g., muscle group. Although sweat may affect the humidity sensors readings, the gel applied to patients' skin is thicker and works well even with sweat. Thus, sweat has minimal effect on the performance of the system.

The humidity sensors 615 may detect the humidity levels where they are located and provide data indicative of the detected humidity levels to the processor 635. For each humidity sensor, the processor 635 may determine whether the received data indicative of the humidity level satisfies (is greater than or equal to) a humidity threshold, in operation S920. If the data indicative of the humidity level satisfies the humidity threshold, the processor 635 may continue to monitor humidity levels between the electrodes of the measurement device 150A and the skin of the user 110, in operation S915.

In operation S920, of the data indicative of the humidity level does not satisfy the humidity threshold, the processor 635 may generate and transmit instructions to the gel dispenser 630 to dispense gel in an area corresponding to the location of the humidity sensor. To dispense gel based on detected humidity levels, the processor 635 may communicate with a storage or memory device accessible by the measurement device 150A. The measurement device 150A may transmit, to the storage/memory device, a query message that includes the detected humidity level and a query for the amount of gel that corresponds to the detected humidity level. The storage/memory device may store a mapping table of humidity levels to amounts of gel to be dispensed. For example, if humidity levels are at a first level, a first amount of gel may be needed to be dispensed per electrode. Accordingly, after receiving data indicative of a humidity level from the measurement device 150A, the storage/memory device may obtain data indicative of the corresponding amount of gel to be dispensed, and may transmit this data to the measurement device 150A. The measurement device 150A may then obtain the amount of gel from a gel storage unit as indicated by the information received from the storage/memory device. Additional details of the gel dispenser 630 are provided with reference to FIG. 10.

According to embodiments a third operational setting that may be employed includes synchronizing gel pumps with one or more exercise patterns. In an embodiment, instead of using a push button, the practitioner could record a video in advance. Then, she/he may replay the video via a computing device 120 along with an input, i.e., a slider at the bottom, to mark the times to pump gel. Initially, the computing device may send the video duration and an identifier (e.g., a video identifier or a user identifier) to the processor 635 or database 140. A practitioner may replay the video and select one or more gel-dispensing times corresponding to events of an exercise program or pattern. In an embodiment, the computing device 120 identifies the gel-dispensing times and stores the associated time marks corresponding to the video (e.g., the video time mark). In an embodiment, the computing device 120 may send the marks with the identifier to update the processor's 635 memory. Alternatively, the computing device 120 may send the marks with the identifier to update the database 140. This embodiment is useful to let practitioners to easily and quickly add gel whenever a challenging or a new set is about to be performed so that the system can pump gel accordingly.

As shown in FIG. 10, an example gel dispenser 630 may include a timer 1010, gel storage area 1020, and a gel pump 1030. The gel dispenser 630 may be connected to one or more gel dispensing tubes 1040. Each of the components of the gel dispenser 630 may be integrated with the gel dispenser 630 as a single unit as shown in FIG. 10, or may be implemented as different units connected to the gel dispenser 630. For example, the gel storage 1020 may be part of the gel disperser 630 or may be a separate unit that may be attached or connected to the gel dispenser 630. The timer 1010 may be a separate unit or a build-in feature on the processor 635.

The gel storage 1020 may be configured to store gel. In general, various types of gel may be used, for example, conductive gel such as Spectra® 360 electrode gel. In some embodiments, the gel storage 1020 may be a cavity in which gel may be stored. In some embodiments, gel storage 1020 may be a replaceable tablet that contains gel and may be replaced when the gel in the replaceable table is finished or depleted. In some embodiments, the gel storage 1020 may be a storage tank with an input port through which gel may be input to fill the tank.

A gel pump 1030 may be connected to the gel storage 1020 and may be configured to obtain gel from the gel storage 1020 and provide the obtained gel to one or more gel tubes 1040. The gel pump 1030 may use pressure differences (e.g., vacuum pressure) to obtain gel from the gel storage 1020. In some embodiments, processor 635 may send instructions to the gel pump 1030. The instructions may include: (i) timing information of when to obtain gel from the gel storage 1020; (ii) quantity information indicating an amount of gel to obtain from the gel storage 1020; and, in some cases (iii) location information indicating a region under the measurement device component 150A where the humidity levels are low.

In some embodiments, the gel pump 1030 may receive a signal from timer 1010 or processor 635 when the set time period expires, as described above in operation S910. In response to receiving the signal from timer 1010 or processor 635, the gel pump 1030 may obtain a determined amount of gel from the gel storage 1020. In the time-based operation mode, the gel pump 1030 may obtain a fixed or predetermined amount of gel for each electrode under which gel is to be dispensed. In the humidity sensor-based operation mode, the processor 635 may provide the gel pump 1030 with information specifying the amount of gel that is to be disposed based on information obtained from the mapping table stored in a storage/memory device as described above. In the synchronization of exercises operation mode, the processor 635 may provide a default or a previous session information indicating the times and amount to dispense gel to configure the gel pump 1030.

In some embodiments, the gel pump 1030 may include a measuring device to measure the amount of gel obtained from gel storage 1020. The measuring device may be located at the interface between the gel pump 1030 and the gel storage 1020, and may detect the amount of gel being passed from the gel storage 1020 to the gel pump 1030. For example, if a determination is made that more gel is needed, the gel pump 1030 may obtain additional gel from the gel storage 1020.

In an embodiment, after obtaining the identified amount of gel, the gel pump 1030 may then dispense the obtained gel through one or more gel tubes 1040 on to the skin 1050 of the user 110 that is underneath an electrode, e.g., electrode 410A. The processor 635 may control one or more valves located at the output ports of the gel dispenser 630 to be open at the time gel is to be dispensed, and closed at a set time period after the opening to allow for sufficient amount of gel to be dispensed. As noted above, in some embodiments, gel may be dispensed on portions of the skin 1050 that are located under the electrodes of the measurement device 150A upon the expiration of a set time period. In some embodiments, gel may be dispensed only at regions where humidity levels were less than the humidity threshold, as detected by the humidity sensors 615. In some embodiments, gel may be dispensed when a practitioner and/or a user requests to pump gel during the sessions; these requests may be saved as the preferred values for subsequent sessions.

In an embodiment, a strong conductive contact between the electrodes of the measurement device 150A and the skin of the user 110 is maintained when obtaining measurements using the measurement device 150A by periodically dispensing gel according to a set time period, dispensing gel according to detected humidity levels, or dispensing gel based on previously requested gel dispensing events. This automated manner of dispensing gel removes the need for medical practitioners to periodically check the humidity levels or to dispense gel and monitor the amount of the gel between the measurement device 150A and the skin of the user 110. Furthermore, gelling the electrodes reduces skin resistance and improves measurement accuracy.

Since most of the operation may be easily added to a database, implementing a machine learning model to predict when a gel pump is needed is useful. With enough collected data, the gel system could be fully automated, offering practitioners and users the ability to perform sessions without the need to apply gel manually or configure the system.

The improved method for dispensing gel along with the above-described method for determining muscle fascicle characteristics provides a novel, cost effective, efficient, and accurate manner of determining a quality and/or responsiveness of muscle fascicles. The small size of the measurement device 150A allows it to be portable and easily transported from one part of the user 110's body to another part, or between different users. While the gel dispensing method and device have been described with respect to measurement device 150A, in general, the gel dispending method may be utilized for any measurement device that uses contacts, such as electrodes, disposed on a user 110's skin.

In an embodiment, the device of the present disclosure may include a detection component (e.g., a hardware component and/or computer-executable instructions executable by a processing device) configured to detect displacement of an electrode and inform a user and/or a practitioner if the system is in need of relocating or repositioning. In an embodiment, the measurement device and some or all of the mentioned systems may share a common casing. The casing may include an inertial measurement unit (IMU) to record location information (e.g., x-y-z axis values) during the initial phase. In the initial phase, the IMU readings of the casing may be saved to the memory of the processor 635, or sent to a computing device 120 or a database 140. The initial values may be used as the expected values for the subsequent sessions. During any setup of subsequent sessions, if the casing placement varies greatly from the initial phase (e.g., 10%), then the system detects that the casing is misplaced and informs the user to change the location. The instructions to position the casing may be displayed inside or around the casing by, e.g., changing colors of a Light-emitting diode (LED). Additionally, the instructions may be displayed on a computing device 120, with color or text combinations to guide the user to move the casing. In an embodiment, the processor 635 and/or computing device 120 may be capable of calculating the difference between the initial x-y-z axes and the current x-y-z readings in order to inform a user to move the casing upward, downward, right and/or left.

In some embodiments, the casing may be replaced with a sleeve (e.g., a cloth sleeve) for greater flexibility and comfort. In some embodiments, the detection component may provide a higher accuracy of electrodes' localization by adding an IMU on each electrode. In order to allow the system to take measurements, a practitioner may notify the system, i.e. using a button, to record the x-y-z measurements of each electrode. The measurements may be sent to the main module, a processor associated with a memory and/or access to a database to store the IMU readings. The processor may take several IMU samples until the readings converge, to avoid a “shaking” error or other noise in the measurement.

In an embodiment, the device of the present disclosure may include a locking sub-system for its electrodes to prevent displacements, as shown in FIGS. 11A-E. In an embodiment, the main module may include, but is not limited to, an EMG, EMS/TENS, IMU, gel dispenser, processor and a locking sub-system around electrodes. The locking sub-system includes one or more structural components configured to operate in two modes: i) a state that reserves or locks an electrode in its place, referred to as a locked state and ii) a state that allows an electrode to move, referred to as an unlocked state. FIG. 11A demonstrates a device in a locked state, where an electrode 1110 is equally spaced between one or more tracks 1100 (e.g., arranged in a zig-zag configuration and referred to as a “zig-zag track”). An electrode 1110 may be surrounded by two walls 1120 to i) prevent it from moving in the locked state and ii) to form an enclosure in the unlocked state which may be easily controlled by an external handle. Additionally, one side of the zig-zag track 1100 may be attached to a movable track 1130 and a surface handle 1140A to extend or reduce the distance between its track. A handle connector 1150 may enable an external source to control the states. In the configuration shown in FIG. 11A, the electrode cannot move in any direction. In an embodiment, this is desirable if a treatment is in progress or a practitioner wants a patient to perform in-home exercises and/or tests while ensuring proper placements.

To switch to an unlocked state, a practitioner may insert a key 1160A into the connector 1150, as shown in FIG. 11B. Consequently, the surface handle 1140B may begin to push the movable track 1130 and a zig-zag track 1100 outward. FIG. 11C depicts the device when the surface handle 1140C is fully pushed out, making the movable track 1130 and a zig-zag track 1100 outward completely. Thus, the device may now move freely since the spacing between the zig-zag track 1100 is extended. Once a practitioner chooses the desired placement (e.g., to the left), the practitioner may take out the key 1160B which pushes the surface handle 1140D inward. As a result, the movable track 1130 and the zig-zag track 1100 may be pushed inward, as shown in FIG. 11D. When the surface handle 1140D is completely inward, as shown in FIG. 11E, the distance between the zig-zag track 1100 is reduced, configuring the electrode 1110 in a locked state.

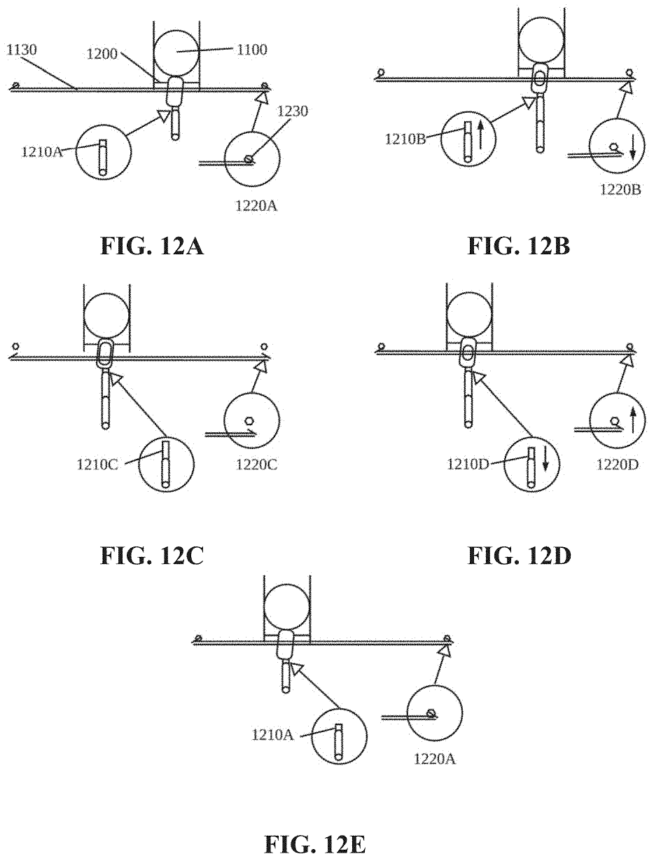

FIGS. 12A-E illustrates the internal process for the locking mechanism. In FIG. 12A, an electrode 1100 position is locked. To enable an external handler to control a direction of the electrode 1100, a handle may be attached to the walls 1120 (shown in FIG. 11A) via horizontal extensions 1200. Since the movable track 1130 has endings 1220A that are within holes 1230, the zig-zag track 1100 and electrode 1110 are locked. FIG. 12B initiates an unlocked state, where a key 1160A may be inserted into a connector 1150. The connector may include a clip 1210A which may be pushed horizontally into the surface handle 1140B to push the movable track 1130 outward of the hole 1230. In FIG. 12C, the clip 1210C has reached the end of its horizontal expansion and the surface handle 1140C is now completely pushed out. Therefore, the zig-zag track 1100 is expanded and the device may become in an unlocked state, allowing an electrode 1110 to change its location. After a practitioner chooses the desired location, a locked state may be initiated. In FIG. 12D, a practitioner may take out the key 1160B which pulls out the clip 1210D from the surface handle 1140D. The movable track 1130 and zig-zag track 1100 may be pushed inward toward the hole 1230. In FIG. 12E, the clip 1210A may be returned to its initial place under the connector 1150, making the track endings 1220A within the hole 1230. FIG. 12E illustrates the electrode 1100 in a locked state with a new position.

In a locked state, electrodes are placed between one or more tracks 1100 and 1150 (e.g., arranged in a zig-zag configuration and referred to as a “zig-zag track”) and surrounded by two walls 1110 to block movement in any direction. In a locked state, electrode placement can be maintained until a practitioner updates or changes the placement for a subsequent session. In an embodiment, the connector 1220 may reside inside the port 1280, as shown in FIGS. 12A and 12B. The strike plate 1120 may touch the zig-zag tracks 1150, but are unable to unlock it since the endings are inside holes 1140.

In an embodiment, to move to an unlocked state, a practitioner or a user may insert a key 1170 into port 1280 to push the connector 1220 forward, as shown on FIG. 11B. The connector 1220 may move the clip 1160 upward by pushing the clip from the button, passing the strike plate 1120, as shown on FIG. 12B. The clip 1160 may begin carrying the zig-zag track 1150 and thus the endings 1140 are no longer reside inside holes 1140, as shown on example 1240. Moreover, the zig-zag track 1150 may move freely inside the space to extend the zig-zag track 1150 to allow electrodes to move. A practitioner or a user may use the key 1170 to move electrodes to the desired location, as shown on FIG. 11C. The strike plate 1120 may be contained or placed next to the electrode and in between the walls 1110, as shown in FIG. 12A. To lock the electrode placement, a practitioner or a user may pull the key 1170 from port 1280, thus bringing back the connector 1220, as shown in FIG. 11D. Due to connector's movement, the clip may be brought downward inside the strike plates 1120. Furthermore, the zig-zag track 1150 may be brought downward, making the endings reside again inside the holes 1140, as shown on FIG. 12B. The electrodes placements have now become locked.

Embodiments and the functional operations regarding the electrodes' placements are presented as possible configurations and not as an inclusive list. For example, another configuration for the lock feature may be to replace the connector, the key and clip with a small motor. The motor may communicate with a processor 635 to unlock electrodes when needed by lifting zig-zag track 1150. The direction of the electrode may be controlled using a knob from a device over a computing device 120. Additionally, a track or more may expand together and move permanently to a new location, allowing the device to change locations in the horizontal and vertical directions.

Overall, once the practitioner initializes or sets up the system, the system is able to lock or remember the desired and targeted muscle fibers. Additionally, it may be possible with the implementation of this system and collecting enough data points about various conditions that the system will be able to know where to place the electrodes according to the patients' condition and the type of muscle fibers targeted.

Embodiments and the functional operations and/or actions described in this specification may be implemented in digital electronic circuitry, or in computer software, firmware, or hardware, including the structures disclosed in this specification and their structural equivalents, or in combinations of one or more of them. Embodiments may be implemented as one or more computer program products, e.g., one or more modules of computer program instructions encoded on a computer readable medium for execution by, or to control the operation of, data processing apparatus. The computer-readable medium may be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program, also known as a program, software, software application, script, or code, may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data in a single file dedicated to the program in question, or in multiple coordinated files. A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random-access memory or both.

Elements of a computer may include a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer may not have such devices. Moreover, a computer may be embedded in another device, e.g., a tablet computer, a mobile telephone, a personal digital assistant (PDA), a mobile audio player, a Global Positioning System (GPS) receiver, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user or driver, embodiments may be implemented on one or more computers having a display device, e.g., a cathode ray tube (CRT), liquid crystal display (LCD), or light emitting diode (LED) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

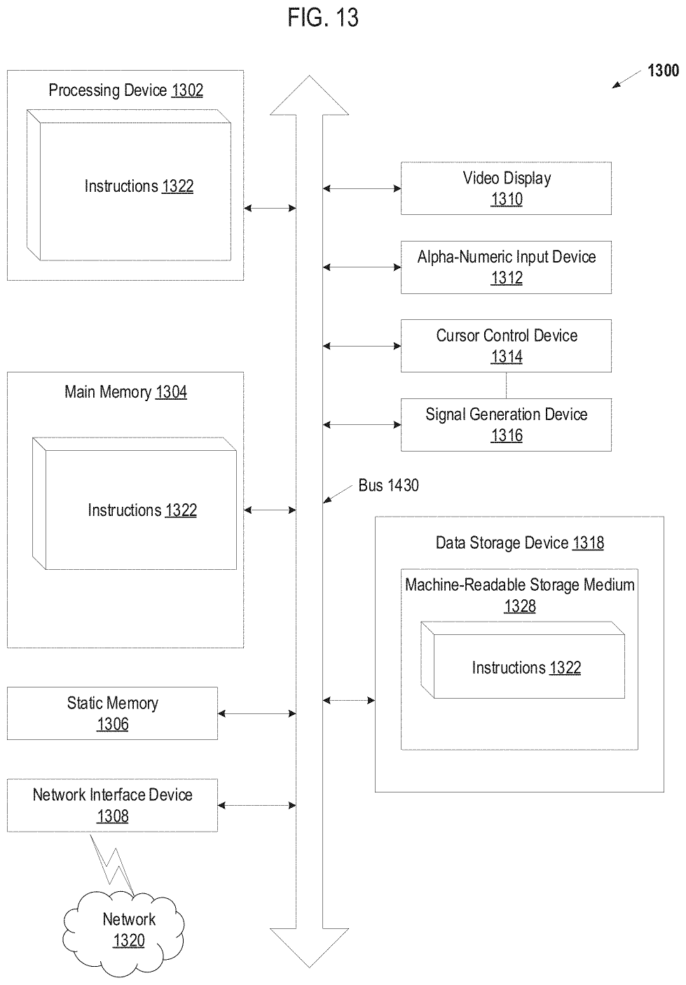

FIG. 13 is a block diagram of an example computer system 1300 that may perform one or more of the operations described herein, in accordance with various implementations. In alternative implementations, the machine may be connected (e.g., networked) to other machines in a LAN, an intranet, an extranet, or the Internet. The machine may operate in the capacity of a server or a client machine in client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1300 includes a processing device (e.g., a processor) 1302, a main memory 1304 (e.g., read-only memory (ROM), flash memory, dynamic random access memory (DRAM) such as synchronous DRAM (SDRAM), double data rate (DDR SDRAM), or DRAM (RDRAM), etc.), a static memory 1306 (e.g., flash memory, static random access memory (SRAM), etc.), and a data storage device 1314, which communicate with each other via a bus 1330.

Processor 1302 represents one or more general-purpose processing devices such as a microprocessor, central processing unit, or the like. More particularly, the processor 1302 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 1302 may also be one or more special-purpose processing devices such as an application specific integrated circuit (ASIC), a field programmable gate array (FPGA), a digital signal processor (DSP), network processor, or the like. The processor 1302 is configured to execute instructions 1322 for performing the operations and steps discussed herein.

The computer system 1300 may further include a network interface device 1304. The computer system 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse), and a signal generation device 1316 (e.g., a speaker).

The data storage device 1314 may include a computer-readable storage medium 1324 on which is stored one or more sets of instructions 1322 (e.g., software) embodying any one or more of the methodologies or functions described herein. The instructions 1322 may also reside, completely or at least partially, within the main memory 1304 and/or within the processor 1302 during execution thereof by the computer system 1300, the main memory 1304 and the processor 1302 also constituting computer-readable storage media. The instructions 1322 may further be transmitted or received over a network 1320 via the network interface device 1308.

In one implementation, the instructions 1322 include instructions associated with programs or modules configured to execute the operations of a measurement device (e.g., measurement device 150 of FIG. 1) and/or a software library containing methods that call the optimization module. While the computer-readable storage medium 1428 (machine-readable storage medium) is shown in an exemplary implementation to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

Some portions of the detailed description have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “collecting”, “establishing”, “generating”, “identifying”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

For simplicity of explanation, the methods are depicted and described herein as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

Certain implementations of the present disclosure also relate to an apparatus for performing the operations herein. This apparatus may be constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

Reference throughout this specification to “one implementation” or “an implementation” means that a particular feature, structure, or characteristic described in connection with the implementation is included in at least one implementation. Thus, the appearances of the phrase “in one implementation” or “in an implementation” in various places throughout this specification are not necessarily all referring to the same implementation. In addition, the term “or” is intended to mean an inclusive “or” rather than an exclusive “or.” Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion.

The terms “first”, “second”, “third”, “fourth”, etc. as used herein are meant as labels to distinguish among different elements and may not necessarily have an ordinal meaning according to their numerical designation.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

While this specification contains many specifics, these should not be construed as limitations on the scope of the disclosure or of what may be claimed, but rather as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable sub-combination. Moreover, although features may be described above as acting in certain combinations and may even be claimed as such, one or more features from a claimed combination may in some cases be excised from the combination, and the claimed combination may be directed to a sub-combination or variation of a sub-combination.

Similarly, while actions are depicted in the drawings in a particular order, this should not be understood as requiring that such actions be performed in the particular order shown or in sequential order, or that all illustrated actions be performed, to achieve desirable results. For example, operations S702-S710 and/or S905-S925 may be executed in various orders and are not limited to the sequential order of the reference numbers assigned to the operations. Moreover, the separation of various system components in the embodiments described above should not be understood as requiring such separation in all embodiments, and it should be understood that the described program components and systems may generally be integrated together in a single software product or packaged into multiple software products.

It will be understood that when an element or layer is referred to as being “on”, “connected to”, or “coupled to” another element, it may be directly on, indirectly on, connected, or coupled to the other element or intervening elements may be present. In contrast, when an element is referred to as being “directly on”, “directly connected to”, or “directly coupled to” another element or layer, there are no intervening elements or layers present.

As used herein, the term “and/or” includes any and all combinations of one or more of the associated listed items. For the purposes of this disclosure, “at least one of X, Y, and Z” may be construed as X only, Y only, Z only, or any combination of two or more items X, Y, and Z (e.g., XYZ, XYY, YZ, ZZ). The phrase “one or more of X, Y, and Z” may be construed as X only, Y only, Z only, or any combination of two or more items X, Y, and Z (e.g., XYZ, XYY, YZ, ZZ).

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another region, layer or section. Thus, a first element, component, region, layer, or section discussed below could be termed a second element, component, region, layer, or section without departing from the disclosed subject matter.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing embodiments only and is not intended to be limiting of the disclosed subject matter. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, operations, elements, and/or components, but do not preclude the presence or addition of one or more other features, integers, operations, elements, components, and/or groups thereof.

Embodiments of the disclosed subject matter are described herein with reference to cross-section illustrations that are schematic illustrations of idealized embodiments (and intermediate structures) of the disclosed subject matter. As such, variations from the shapes of the illustrations as a result, for example, of manufacturing techniques and/or tolerances, are to be expected. Thus, embodiments of the disclosed subject matter should not be construed as limited to the particular shapes of regions illustrated herein but are to include deviations in shapes that result, for example, from manufacturing.

It will be apparent to those skilled in the art that various modifications and variations may be made without departing from the spirit or scope of the disclosed subject matter. Thus, it is intended that the present disclosure cover the modifications and variations of the disclosed subject matter provided they come within the scope of the appended claims and their equivalents.

What is claimed is:

1. A system comprising:
a first set of electrodes disposed in a first region corresponding to a set of a plurality of muscle fascicles of a subject;
a second set of electrodes disposed in the first region corresponding to the set of the plurality of muscle fascicles;
a stimulation device configured to:
transmit a first signal to a first electrode of the first set of electrodes; and
transmit a second signal to a second electrode of the first set of electrodes, the second signal having less power than the first signal;
a sensing device configured to detect a first response signal using a first detecting electrode in response to transmission of the first signal and the second signal; and
a processor configured to determine at least one feature of a first muscle fascicle among the set of the plurality of muscle fascicles based on the first response signal.

2. The system of claim 1, wherein the at least one feature comprises at least one of an indication of a responsiveness of a nerve associated with the first muscle fascicle or an indication of a quality of the first muscle fascicle.

3. The system of claim 1, further comprising:
a second detecting electrode configured to detect a second response signal in response to transmission of the first signal and the second signal, wherein each of the first detecting electrode and the second detecting electrode comprises a pair of detecting electrodes; and
a reference electrode configured to be disposed in a second region that is different than the first region, the reference electrode to provide a reference signal to the sensing device, wherein the sensing device is configured to aggregate the reference signal, the first response signal, and the second response signal.

4. The system of claim 3, further comprising a locking sub-system operable to transition the first electrode between a locked state and an unlocked state.

5. The system of claim 4, the locking sub-system comprising:
a first track, and
a second track opposing the first track, wherein the second track is configured to move toward the first track to transition the first electrode in the locked state between the first track and the second track.

6. The system of claim 5, the locking sub-system further comprising a handle configured to engage with the second track to adjust a position of the second track relative to the first track.

7. The system of claim 6, the locking sub-system further comprising a key configured to couple with the handle to control operation of the handle to transition the first electrode between the locked state and the unlocked state.

8. The system of claim 1, wherein the first set of electrodes in the first detecting electrode are configured to be positioned proximate to a first end of the set of a plurality of muscle fascicles located on a first end of the set of the plurality of muscle fascicles; and wherein the second set of electrodes in the second detecting electrode are configured to be positioned proximate to a second end of the set of a plurality of muscle fascicles.

9. The system of claim 1, further comprising:
a gel dispenser configured to dispense gel at a location between the first set of electrodes and an epidermis of the subject.

10. The system of claim 9, wherein the gel dispenser is configured to dispense gel at a plurality of instances according to a selected frequency of time.

11. The system of claim 9, further comprising:
a humidity sensor configured to determine a humidity level value in an area proximate to the first region, wherein the gel dispenser is configured to dispense gel in response to determining the humidity level does not satisfy a humidity level threshold.

12. The system of claim 9, wherein the gel dispenser is configured to dispense gel at a plurality of instances corresponding to an exercise program.

13. A method comprising:
transmitting a first signal to a first electrode of a first set of electrodes, the first electrode disposed proximate to a first muscle fascicle;
transmitting a second signal to a second electrode of the first set of electrodes, the second electrode disposed proximate to a set of additional muscle fascicles adjacent to the first muscle fascicle, wherein the second signal has less power than the first signal;
detecting, using a second set of electrodes, a first response signal in response to transmission of the first signal and the second signal;
detecting, using the second set of electrodes, a second response signal in response to transmission of the first signal and the second signal;
receiving a reference signal from a reference electrode disposed in a region other than where the muscle fascicles are located, wherein determining the first feature of the muscle fascicle comprises determining the at least one feature based on the reference signal, the first response signal, and the second response signal and
determining, by a processor, a first feature of the muscle fascicle based at least in part on the first response signal, the second response signal, and the reference signal.

14. The method of claim 13, wherein the first feature comprises at least one of an indication of a responsiveness of a nerve associated with the first muscle fascicle or an indication of a quality of the first muscle fascicle.

15. The method of claim 13, further comprising:
a first track, and
a second track opposing the first track, wherein the second track is configured to move toward the first track to transition the first electrode in the locked state between the first track and the second track.

16. The method of claim 13, further comprising:
transmitting a third signal to a third electrode of the first set of electrodes;
transmitting a fourth signal to a fourth electrode of the first set of electrodes, wherein the fourth signal has less power than the third signal;
receiving a third response signal at a first pair of electrodes of the second set of electrodes;
receiving a fourth response signal at a second pair of electrodes in the second set of electrodes; and
determining at least one feature of the first muscle fascicle based on the first response signal, the second response signal, the third response signal, and the fourth response signal.

17. The method of claim 16, further comprising:
transmitting two or more test signals via the first set of electrodes;
receiving a first test response signal via the second set of electrodes;
displaying amplitude information and phase information of the first test response signal; and
updating the displayed amplitude and phase information in response to movements of the first set of electrodes and the second set of electrodes.

18. The method of claim 13, further comprising:
receiving, from at least one sensor, data indicative of a humidity level in an area associated with the first electrode;
determining the humidity level fails to satisfy a humidity level threshold; and
controlling a gel dispenser to dispense a first amount of gel in at least a portion of the area in response to the data indicative of the humidity level failing to satisfy the humidity level threshold.

19. The method of claim 18, further comprising controlling one or more valves of the gel dispenser in the portion of the area to establish an open position; and
controlling the gel dispenser to dispense the amount of gel corresponding to the humidity level through the one or more valves of the gel dispenser.

20. The method of claim 13, further comprising:
tracking an amount of lapsed time since a previous disposition of gel by the gel dispenser;
determining the amount of lapsed time satisfies a time period threshold; and
dispensing gel in response to the amount of time satisfying the time period threshold.
